(19) United States
(12) Patent Application Publication
(10) Pub. No.: US 2026/0224212 A1
(43) Pub. Date: Aug. 6, 2026

(54) TISSUE REPAIR DEVICES, SYSTEMS, AND METHODS

(71) Applicant: Integrity Medical Services Inc., Yorba Linda, CA (US)

(72) Inventor: Nirav H. Amin, Yorba Linda, CA (US)

(21) Appl. No.: 19/462,762

(22) Filed: Jan. 28, 2026

Related U.S. Application Data
(60) Provisional application No. 63/752,457, filed on Jan. 31, 2025.

(51) Int. Cl. A61B 17/04; A61B 17/17; A61B 17/00

(52) U.S. Cl. CPC A61B 17/0469; A61B 17/1742; A61B 17/1796; A61B 2017/00367; A61B 2017/047

(57) ABSTRACT

A snare device with a retractable snare to retrieve a suture. The snare can have a piercing component on the distal end, which can be two wires twisted together. The piercing end can allow the snare to pierce through tissue to reach a target site. The snare can be large enough to extend through a tunnel in bone and retrieve a suture on the other side.

### INCORPORATION BY REFERENCE TO ANY PRIORITY APPLICATIONS

This application claims priority to the U.S. Provisional Application 63/752457, filed January 31, 2025, which is incorporated by reference herein in its entirety.

### TECHNICAL FIELD

The present disclosure relates to the field of suture retriever methods and devices, and more particularly to arthroscopic ligament repair and/or reconstruction surgery.

### BACKGROUND

Desired outcomes for arthroscopic ligament reconstruction or repair surgery are generally achieved by establishing the proper shape and placement of torn tissue. While performing such surgery, a surgeon typically makes a small incision in a patient’s skin near the surgical site (e.g., a bone joint) to allow a drill assembly to be placed in the bone joint to create tunnels through the patient’s bone, through which the surgeon can reconstruct and/or repair the torn tissue.

For a meniscal root repair, for example, one approach is to create a tunnel through the patient’s tibia for the passing of a suture or wire. The suture is passed to the location of repair, and the meniscus is repaired with a button, anchor, or other suitable method of fixation. In some instances, a suture retriever having a snare is used to capture a suture from one side of the bone tunnel and pull it through the tunnel. In this use and other uses for such suture retrievers, the snare must pass through tissue or other structures. Current snares can have dull distal ends, such that it can be difficult to pass through obstacles in the patient’s body.

Accordingly, there is a need for suture snares that can pierce through obstacles in a patient’s body. There is a need for large suture snares with piercing features on the distal end to improve maneuverability and effectiveness for capturing sutures.

### SUMMARY

In some examples, the suture retriever described herein can include: a main body; a snare loop configured to extend from a distal end of the main body, the snare loop including a piercing element on a distal end of the snare loop; and an actuator configured to control extension and retraction of the snare loop.

In some examples, the snare loop is extendable 8-10 cm beyond a distal end of the main body. In some examples, the piercing element includes a plurality of monofilaments twisted together. In some examples, the piercing element includes a plurality of monofilaments, wherein a distal tip of each monofilament is welded together. In some examples, the piercing element is configured to cut through tissue to position the snare loop. In some examples, the main body is configured to couple with a repair guide such that the snare loop can extend distally through the repair guide. In some examples, the device can include a base portion proximal to the main body. In some examples, the actuator is a sliding component on the base portion. In some examples, the actuator is a separable portion of the base portion movable in a proximal and distal direction, the separable portion coupled with a pushing rod configured to move the snare loop. In some examples, the piercing element is a needle.

In some examples, the method for repairing a hip described herein can include: drilling, with a drill bit, a tunnel in a bone; advancing a snare device into the tunnel in the bone, the snare device including a body portion and a snare loop, the snare loop including a piercing element on a distal end of the snare loop; exposing the snare loop from a distal end of the body portion of the snare device such that the piercing element pierces tissue; advancing a suture through the snare loop; and retracting the snare loop to capture the suture.

In some examples, the method can include retracting the snare device to pull the suture through the tunnel in the bone. In some examples, the piercing element includes a plurality of monofilaments twisted together. In some examples, exposing the snare loop includes extending the snare loop 8-10 cm beyond a distal end of the body portion of the snare device. In some examples, retracting the snare loop to capture the suture includes pulling the suture into the body portion of the snare device. In some examples, the piercing element includes a plurality of monofilaments, wherein a distal tip of each monofilament is welded together. In some examples, the method can include extending the snare loop through a tissue repair guide, the tissue repair guide configured to guide the snare loop into the tunnel in the bone.

In some examples, the systems for retrieving a suture described herein can include: a suture retriever device including: a main body; a snare loop configured to extend from a distal end of the main body, the snare loop including a piercing element on a distal end of the snare loop; and an actuator configured to control extension and retraction of the snare loop; and a tissue repair guide including a drill bit, the tissue repair guide configured to guide the snare loop through the drill bit, wherein a distal end of the main body of the suture retriever device is configured to couple with the tissue repair guide.

In some examples, the snare loop is extendable 8-10 cm beyond a distal end of the main body. In some examples, the piercing element includes a plurality of monofilaments twisted together. In some examples, the piercing element includes a plurality of monofilaments, wherein a distal tip of each monofilament is welded together. In some examples, the kit for repairing a hip described herein can include: a drill bit; and a retractable snare device including a snare loop.

### BRIEF DESCRIPTION OF THE DRAWINGS

FIG. 1A illustrates an example of a suture retrieving snare device with a snare loop.

FIG. 1B illustrates the suture retrieving snare device of FIG. 1A with the snare loop partially retracted.

FIG. 1C illustrates the suture retrieving snare device of FIG. 1A with the snare loop further retracted.

FIG. 1D illustrates the suture retrieving snare device of FIG. 1A with the snare loop completely retracted.

FIG. 2A illustrates an example of a suture retrieving snare device.

FIG. 2B illustrates the suture retrieving snare device of FIG. 2A with the snare loop partially retracted.

FIG. 2C illustrates the suture retrieving snare device of FIG. 2A with the snare loop further retracted.

FIG. 2D illustrates the suture retrieving snare device of FIG. 2A with the snare loop completely retracted.

FIG. 2E is an exploded view of the suture retrieving snare device of FIG. 2A.

FIG. 3 shows an example of a kit with the suture retrieving snare device of FIG. 2A.

FIG. 4A shows the example of a snare device of FIG. 1A with the snare retracted in use with tissue repair guide.

FIG. 4B shows the example of a snare device of FIG. 1A with the snare extended in use with tissue repair guide of FIG. 4A.

### DETAILED DESCRIPTION

Embodiments disclosed herein relate to devices, systems, and methods of a snare for suture retrieval. Certain embodiments of the suture retrieving devices herein may be combined with other surgical tools, such as a tissue repair guide, drill, or other suitable devices.

### Suture Retrieving Snare

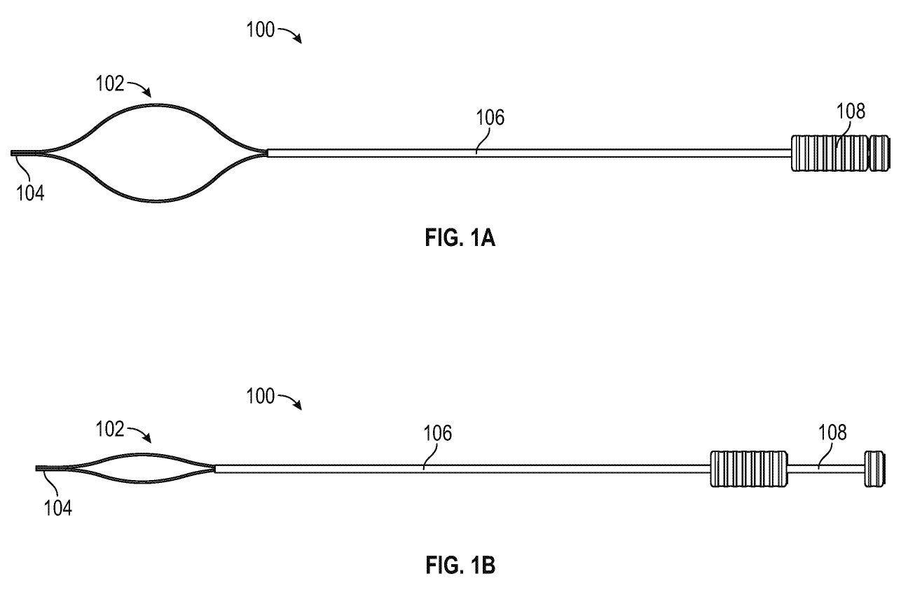

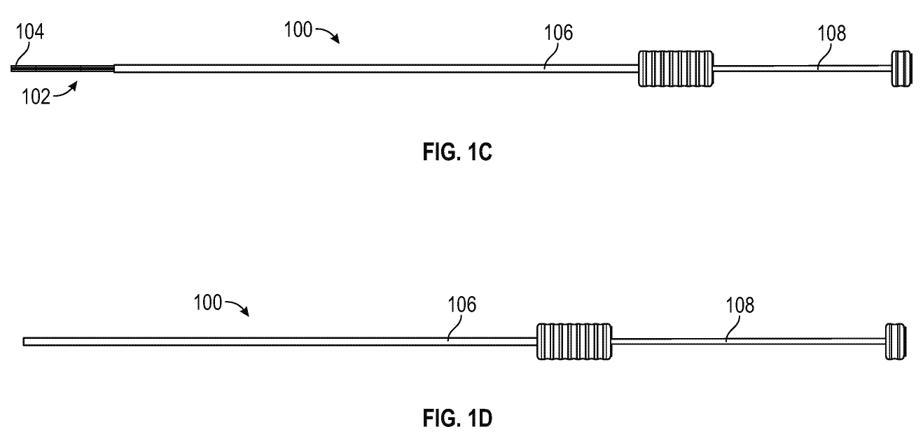

FIG. 1A illustrates an example of a suture retrieving snare device 100 with a snare loop 102. FIG. 1B illustrates the suture retrieving snare device 100 of FIG. 1A with the snare loop 102 partially retracted. FIG. 1C illustrates the suture retrieving snare device 100 of FIG. 1A with the snare loop 102 further retracted. FIG. 1D illustrates the suture retrieving snare device 100 of FIG. 1A with the snare loop 102 completely retracted.

In certain examples, the snare loop 102 can be a monofilament. Both ends of the monofilament can be contained within a body portion 106, or main body, of the snare device 100. The snare loop 102 can have a closed distal end. The distal end of the snare loop 102 can be retractable and extendible from the distal end of the body portion 106. The snare loop 102 can include a piercing component 104 on the distal end. The piercing element 104 may be formed by twisting two or more wires together. The piercing element 104 may be formed by twisting two or more filaments, for example monofilaments, together. In some examples, the monofilaments can be welded together. For example, the monofilaments can be welded together at the distal tip of each monofilament. In some examples, the piercing element 104 can be comprised of 2 monofilaments. In some examples, the piercing element 104 can be comprised of 2-5 monofilaments. In some examples, the piercing element 104 can be comprised of 1-8 monofilaments.

In examples, the piercing element 104 may allow the snare loop 102 to be passed easily through tissue or other obstacles in a patient’s body. In some examples, the piercing element 104 can be sharp enough to cut through soft tissue, but not sharp enough to cut through hard tissue. The snare loop 102 can easily grasp sutures. The snare device 100 can be used for open and/or arthroscopic surgery. In some examples, the piercing element 104 can be a needle.

In some examples, the snare loop 102 may be large enough to extend through a tunnel in bone from a first end and expand beyond a second end of the tunnel. The snare loop 102 can extend a length of at least 8 cm and/or less than or equal to 10 cm past the body portion 106. The snare loop 102 can extend a length of at least 5 cm and/or less than or equal to 15 cm past the body portion 106. The snare loop 102 can extend a length of at least 2 cm and/or less than or equal to 20 cm past the body portion 106.

In some examples, the body portion 106 can include an actuator 108 for extending and retracting the snare loop 102. The actuator 108 can control extension and retraction of the snare loop 102. As shown in FIG. 1A, the snare loop 102 can be completely extended from the distal end of the body portion 106. As shown in FIG. 1B, the snare loop 102 can be partially extended from the body portion 106 such that the snare loop 102 expands only partially radially outward. As shown in FIG. 1C, the snare loop 102 can be slightly extended from the body portion 106 such that the snare loop 102 extends distally but not radially outward. As shown in FIG. 1D, the snare loop 102 can be completely retracted into the body portion 106. Advantageously, the piercing element 104 can cut through material when the snare loop 102 is slightly, partially, or completely extended. In some examples, as shown in FIG. 1C, when the snare loop 102 is slightly extended, the piercing element 104 can cut through tissue effectively due to the straight formation of the snare loop 102 exerting force directly in the distal direction. The actuator 108 can be a slider, a pin, or a button on the proximal end of the body portion 106. Pulling the actuator 108 proximally can retract the snare loop 102 while pushing the actuator 108 distally can extend the snare loop. Applying force to the actuator 108 to extend the snare loop 102 can cause the piercing element 104 to pierce tissue.

In some examples, the distal end of the snare device 100 can be inserted into a bone or tissue site. For example, a user can form a hole in a bone and position the distal end of the snare device 100 in the tunnel. The snare loop 102 can be exposed from the distal end of the body portion 106 such that the piercing element 104 pierces tissue to reach a target site. The snare loop 102 can expand radially as it is exposed from the body portion 106. The snare loop 102 can retract into the body portion 106 with the suture positioned radially within the snare loop 102. The snare device 100 can then be retracted to carry the suture through the tunnel and/or from the target site.

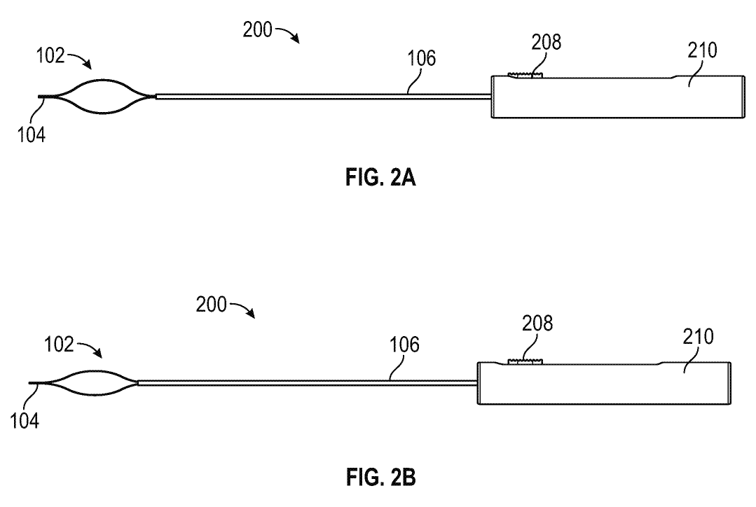

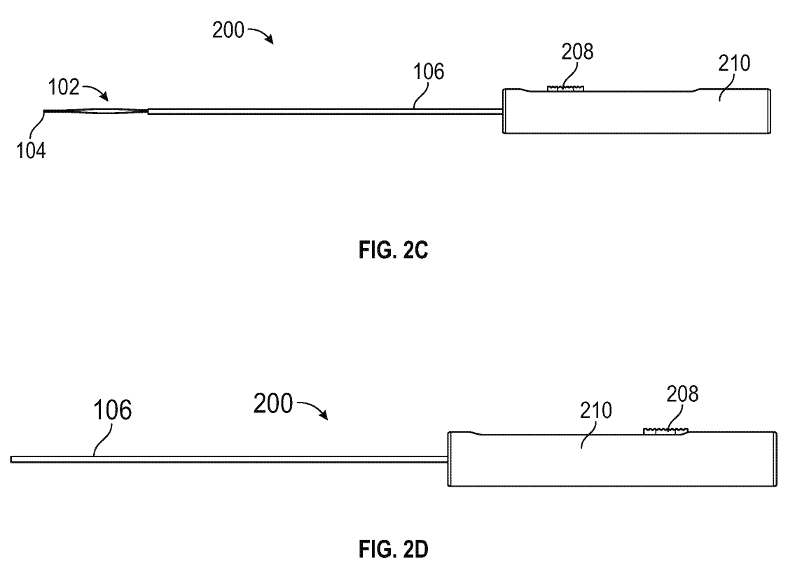

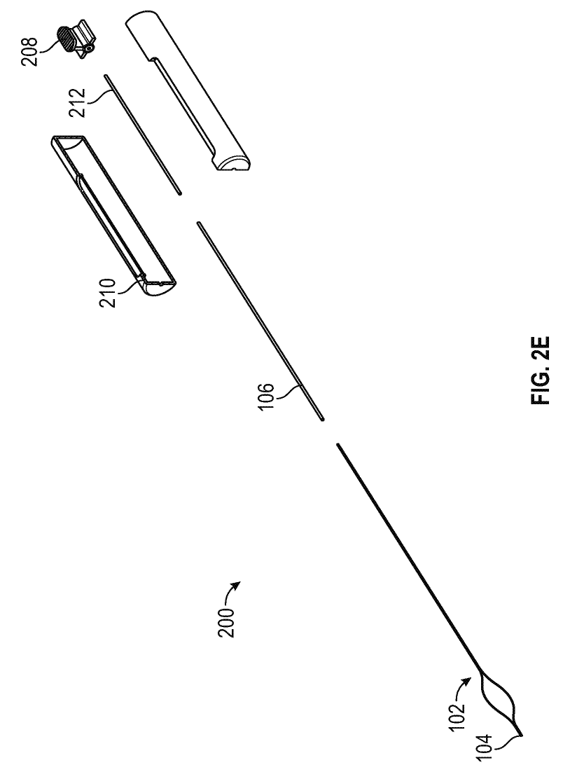

FIG. 2A illustrates another example of a suture retrieving snare device 200. FIG. 2B illustrates the suture retrieving snare device 200 of FIG. 2A with the snare loop 102 partially retracted. FIG. 2C illustrates the suture retrieving snare device 200 of FIG. 2A with the snare loop 102 further retracted. FIG. 2D illustrates the suture retrieving snare device 200 of FIG. 2A with the snare loop 102 completely retracted. FIG. 2E is an exploded view of the suture retrieving snare device 200 of FIG. 2A.

In some examples, the suture retrieving snare device 200 can include all the features of the suture retrieving snare device 100 of FIG. 1A.

In some examples, the snare device 200 can include a base portion 210 proximal to the body portion 106. An actuator 208, for example a sliding mechanism, can be on the base portion 210. In some examples, the actuator 208 can be a switch, a trigger, or a button. Moving the actuator 208 along the base portion 210, for example along a groove, can cause a pushing rod 212 to move distally and/or proximally. The pushing rod 212 can move the snare 102 distally and/or proximally. The pushing rod 212 can be within the body portion 106 and/or the base portion 210. The pushing rod 212 can be a tube. In some examples, the actuator 208 can be a motor, solenoid, pneumatic cylinder, hydraulic actuator, linear actuator, rack and pinion, cam mechanism, ball screw, gear train, voice coil actuator, pneumatic piston, rotating drum, belt drive, crankshaft, lead screw, and/or piezoelectric actuator.

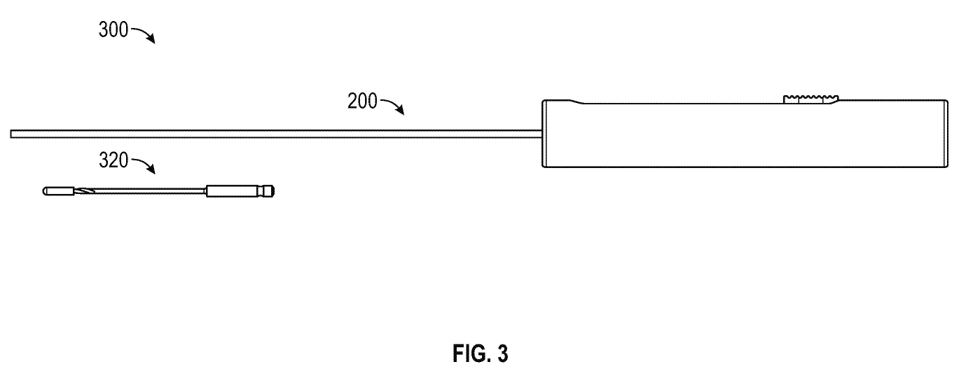

FIG. 3 shows an example of a kit 300 with the suture retrieving snare device 200 of FIG. 2A and drill bit 320.

In some examples, the drill bit 320 can be used to form a tunnel in tissue, for example bone. The snare device 200 can be used to extend a snare through the tunnel to retrieve a suture on the other side of the tunnel. In some examples, the snare device 100 of FIG. 1A may be included in the kit 300. The kit 300 can include a suture retrieval hook, handle, trocar, guide wire, cannula, suture passer, knot pusher, suture grasper, forceps, bone drill, sleeve, depth gauge, scissor, needle holder, retrieval catheter, suture tensioner, endoscopic camera, light source, suction device, and/or fluid pump.

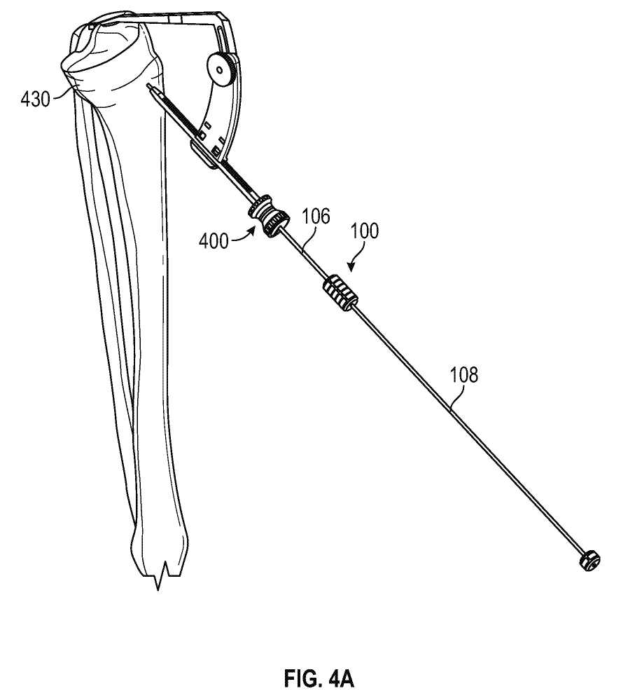

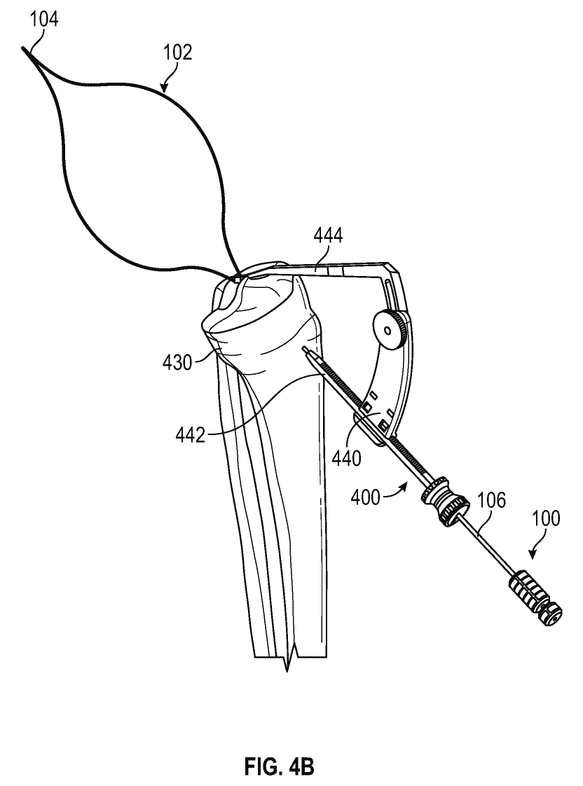

FIG. 4A shows the example of a snare device 100 of FIG. 1A with the snare 102 retracted in use with tissue repair guide 400. FIG. 4B shows the example of a snare device 100 of FIG. 1A with the snare 102 extended in use with tissue repair guide 400 of FIG. 4A.

In some examples, the snare device 200 of FIG. 2A may be used with the tissue repair guide 400. The tissue repair guide 400 may include any and/or all of the features described in U.S. Non-Provisional Application No. 18/770306, filed July 11, 2024, which is incorporated by reference herein in its entirety.

In some examples, the tissue repair guide 400 can be used to form a tunnel in bone 430. The tissue repair guide 400 can include a drill bit. The tissue repair guide 400 can guide the snare 102, or snare loop through the drill bit. A distal end of the body portion 106 of the suture retriever device 100 can couple with the tissue repair guide 400. Advantageously, this can allow the snare 102 to be directed toward the tunnel in the bone. The snare device 100 can be inserted into the tissue repair guide 400 after the tunnel is formed in the bone 430. In some examples, the snare device 100 can be inserted into the tissue repair guide 400 before the tunnel is formed in the bone 430. The snare 102 can be extended through the tissue repair guide 400 and/or tunnel in bone 430 using the actuator 108. Once the snare 102 exits the tunnel, the snare 102 can expand radially. A suture can be passed through the snare 102. The snare 102 can be retracted to pull the suture through the bone 430, the tissue repair guide 400, and/or the snare device 100. When retracted, the snare 102 can contract radially inward to engage the suture. Further retracting the snare 102 can pull the suture into the snare device 100, for example the body portion 106. Once the suture has been captured, the tissue repair guide 400 and/or the snare device 100 can be retracted to pull the suture out of the tunnel in the bone 430. In some examples, the tissue repair guide 400 can be a meniscus repair guide or an ACL repair guide.

In some examples, the repair guide 400 may include a guide handle 440, a drill guide 442, and a bone guide member 444. The bone guide member 444 may also be referred to as a reference member 444.

In some examples, the guide handle 440 may engage the drill guide 442. For example, a through hole in the guide handle 440 may allow engagement of the guide handle 440 and the drill guide 442. A securement feature may be used to secure, connect, fasten, or otherwise couple the guide handle 440 and the drill guide 442.

In some examples, the guide handle 440 may also engage the reference member 444. The securement feature may be used to secure, connect, fasten, or otherwise couple the guide handle 440 and the reference member 444. In some examples, securement features may be screws. For example, in some examples, securement features may be knurled thumbscrews.

In some examples, a repair guide 400 configured of individual, or modular, components may advantageously promote interchangeability, replaceability, and disposability of the components of the repair guide 400.

In some examples, the reference member 444 may include a first arm and a second arm. The first arm and the second arm may define an angle. The angle can be any desired angle to provide optimal anatomic fixation of the meniscus root.

In some examples, the first arm of the reference member 444 may be configured to engage the handle 440. In some examples, the first arm of the reference member 444 may engage a slot of the handle 440. A securement feature may be used to secure, connect, fasten, or otherwise couple the first arm of the reference member 444 to the handle 440.

In some examples, the second arm of the reference member 444 may be configured to engage a patient’s anatomical features. For example, the second arm may be configured to engage the patient’s tibia. The second arm 334 may be configured to engage the tibial plateau of the patient’s tibia. Engagement between the second arm and the patient’s tibia may advantageously help to align subsequent meniscal root repair procedures. Advantageously, this can be used to align the suture retriever 100 with the position of the suture.

In some examples, the second arm may include a guide assistance feature at the tip of the second arm. For example, the guide assistance feature may include a fork feature, such as a forked tip at the tip of the second arm. The guide assistance feature may be configured to engage an anatomical feature of the patient. For example, the guide assistance feature may be sized and shaped such that it engages a portion of the patient’s tibia. The guide assistance feature having a forked feature may advantageously give the surgeon improved visual access while placing the meniscal root guide and performing meniscal root repair procedures. For example, a forked feature at the tip of the second arm may give the surgeon visual access from an intra articular camera view. The guide assistance feature can also be a circular feature. In some examples, the snare loop 102 can be directed to exit the bone tunnel at a position between the prongs of the fork feature or in the middle of the circular feature.

In some examples, the reference member may comprise a rigid material. The reference member may be substantially flat, or thin, which may advantageously reduce the risk of injury or damage to a patient when the reference member is inserted into the patient.

In some examples, advantageously, the piercing element 104 of the snare 102 can pierce tissue and/or obstructions on the far side of the tunnel in the bone 430. This can allow the snare 102 to avoid being obstructed during expansion.

### Non-Limiting Examples

Example 1. A suture retriever, comprising: a main body; a snare loop configured to extend from a distal end of the main body, the snare loop comprising a piercing element on a distal end of the snare loop; and an actuator configured to control extension and retraction of the snare loop.

Example 2. The suture retriever of Example 1, wherein the snare loop is extendable 8-10 cm beyond a distal end of the main body.

Example 3. The suture retriever of any one of Examples 1 or 2, wherein the piercing element comprises a plurality of monofilaments twisted together.

Example 4. The suture retriever of any one of Examples 1-3, wherein the piercing element comprises a plurality of monofilaments, wherein a distal tip of each monofilament is welded together.

Example 5. The suture retriever of any one of Examples 1-4, wherein the piercing element is configured to cut through tissue to position the snare loop.

Example 6. The suture retriever of any one of Examples 1-5, wherein the main body is configured to couple with a repair guide such that the snare loop can extend distally through the repair guide.

Example 7. The suture retriever of any one of Examples 1-6, further comprising a base portion proximal to the main body.

Example 8. The suture retriever of Example 7, wherein the actuator is a sliding component on the base portion.

Example 9. The suture retriever of Example 7, wherein the actuator is a separable portion of the base portion movable in a proximal and distal direction, the separable portion coupled with a pushing rod configured to move the snare loop.

Example 10. The suture retriever of any one of Examples 1-9, wherein the piercing element is a needle.

Example 11. A method for repairing a hip, the method comprising: drilling, with a drill bit, a tunnel in a bone; advancing a snare device into the tunnel in the bone, the snare device comprising a body portion and a snare loop, the snare loop comprising a piercing element on a distal end of the snare loop; exposing the snare loop from a distal end of the body portion of the snare device such that the piercing element pierces tissue; advancing a suture through the snare loop; and retracting the snare loop to capture the suture.

Example 12. The method of Example 11, further comprising retracting the snare device to pull the suture through the tunnel in the bone.

Example 13. The method of any one of Examples 11 or 12, wherein the piercing element comprises a plurality of monofilaments twisted together.

Example 14. The method of any one of Examples 11-13, wherein exposing the snare loop comprises extending the snare loop 8-10 cm beyond a distal end of the body portion of the snare device.

Example 15. The method of any one of Examples 11-14, wherein retracting the snare loop to capture the suture comprises pulling the suture into the body portion of the snare device.

Example 16. The method of any one of Examples 11-15, wherein the piercing element comprises a plurality of monofilaments, wherein a distal tip of each monofilament is welded together.

Example 17. The method of any one of Examples 11-16, further comprising extending the snare loop through a tissue repair guide, the tissue repair guide configured to guide the snare loop into the tunnel in the bone.

Example 18. A system for retrieving a suture comprising: a suture retriever device comprising: a main body; a snare loop configured to extend from a distal end of the main body, the snare loop comprising a piercing element on a distal end of the snare loop; and an actuator configured to control extension and retraction of the snare loop; and a tissue repair guide comprising a drill bit, the tissue repair guide configured to guide the snare loop through the drill bit, wherein a distal end of the main body of the suture retriever device is configured to couple with the tissue repair guide.

Example 19. The system of Example 18, wherein the snare loop is extendable 8-10 cm beyond a distal end of the main body.

Example 20. The system of any one of Examples 18 or 19, wherein the piercing element comprises a plurality of monofilaments twisted together.

Example 21. The system of any one of Examples 18-20, wherein the piercing element comprises a plurality of monofilaments, wherein a distal tip of each monofilament is welded together.

Example 22. A kit for repairing a hip, the kit comprising: a drill bit; and a retractable snare device comprising a snare loop.

Clause 23. A suture retriever, comprising: a main body; and a snare loop configured to extend from a distal end of the main body, the snare loop comprising a piercing element on a distal end of the snare loop, wherein the snare loop comprises a monofilament.

Clause 24. The suture retriever of clause 23, wherein the snare loop is extendable 8-10 cm beyond a distal end of the main body.

Clause 25. The suture retriever of any one of clauses 23 or 24, wherein the piercing element comprises a plurality of ends of the monofilament twisted together.

Clause 26. The suture retriever of any one of clauses 23-25, wherein the piercing element comprises a plurality of monofilaments, wherein a distal tip of each monofilament is welded together.

Clause 27. The suture retriever of any one of clauses 23-26, wherein the piercing element is configured to cut through tissue to position the snare loop.

Clause 28. The suture retriever of any one of clauses 23-27, wherein the main body is configured to couple with a repair guide such that the snare loop can extend distally through the repair guide.

Clause 29. The suture retriever of any one of clauses 23-28, further comprising a base portion proximal to the main body.

Clause 30. The suture retriever of clause 29, further comprising a sliding component on the base portion.

Clause 31. The suture retriever of clause 29, further comprising a separable portion of the base portion movable in a proximal and distal direction, the separable portion coupled with a pushing rod configured to move the snare loop.

Clause 32. The suture retriever of any one of clauses 23-31, wherein the piercing element is a needle.

Clause 33. A method for repairing tissue, the method comprising: advancing a snare loop from a distal end of a body portion of a snare device such that a piercing element on a distal end of the snare loop pierces a tissue, wherein the snare loop comprises a monofilament; advancing a fixation material through the snare loop; and after advancing the fixation material through the snare loop, retracting the snare loop at least partially into the body portion of the snare device to capture the fixation material.

Clause 34. The method of clause 33, further comprising advancing the body portion of the snare device into a tunnel in a bone.

Clause 35. The method of clause 34, further comprising, after advancing the body portion of the snare device into the tunnel in the bone and retracting the snare loop at least partially into the body portion of the snare device to capture the fixation material, retracting the snare device to pull the fixation material through a tunnel in the bone.

Clause 36. The method of any one of clauses 33-35, wherein the piercing element comprises a plurality of ends of the monofilament twisted together.

Clause 37. The method of any one of clauses 33-36, wherein exposing the snare loop comprises extending the snare loop 8-10 cm beyond a distal end of the body portion of the snare device.

Clause 38. The method of any one of clauses 33-37, wherein retracting the snare loop to capture the fixation material comprises pulling the fixation material into the body portion of the snare device.

Clause 39. The method of any one of clauses 33-38, wherein the piercing element comprises a plurality of monofilaments, wherein a distal tip of each monofilament is welded together.

Clause 40. The method of any one of clauses 33-39, further comprising extending the snare loop through a tissue repair guide, the tissue repair guide configured to guide the snare loop.

Clause 41. The method of any one of clauses 33-40, wherein advancing the snare loop from the distal end of the body portion comprises moving a sliding actuator on the body portion.

Clause 42. The method of any one of clauses 33-41, wherein advancing the snare loop from the distal end of the body portion comprises moving a movable portion of the body portion with respect to a main portion of the body portion.

Clause 43. A method for repairing tissue, the method comprising: advancing a snare loop from a distal end of a body portion of a snare device such that a piercing element on a distal end of the snare loop pierces a tissue; advancing a suture through the snare loop; and after advancing the suture through the snare loop, retracting the snare loop at least partially into the body portion of the snare device to capture the suture.

Clause 44. The method of clause 43, further comprising advancing the body portion of the snare device into a tunnel in a bone.

Clause 45. The method of clause 44, further comprising, after advancing the body portion of the snare device into the tunnel in the bone and retracting the snare loop at least partially into the body portion of the snare device to capture the suture, retracting the snare device to pull the suture through a tunnel in the bone.

Clause 46. The method of any one of clauses 43-45, wherein the piercing element comprises a plurality of monofilaments twisted together.

Clause 47. The method of any one of clauses 43-46, wherein exposing the snare loop comprises extending the snare loop 8-10 cm beyond a distal end of the body portion of the snare device.

Clause 48. The method of any one of clauses 43-47, wherein retracting the snare loop to capture the suture comprises pulling the suture into the body portion of the snare device.

Clause 49. The method of any one of clauses 43-48, wherein the piercing element comprises a plurality of monofilaments, wherein a distal tip of each monofilament is welded together.

Clause 50. The method of any one of clauses 43-49, further comprising extending the snare loop through a tissue repair guide, the tissue repair guide configured to guide the snare loop.

Clause 51. The method of any one of clauses 43-50, wherein advancing the snare loop from the distal end of the body portion comprises moving a sliding actuator on the body portion.

Clause 52. The method of any one of clauses 43-51, wherein advancing the snare loop from the distal end of the body portion comprises moving a movable portion of the body portion with respect to a main portion of the body portion.

### Other Variations

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

Conditional language used herein, such as, among others, “can,” “could”, “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” "below," and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component or directly connected to the second component. As used herein, the term “plurality” denotes two or more. For example, a plurality of components indicates two or more components.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Headings are included herein for reference and to aid in locating various sections. These headings are not intended to limit the scope of the concepts described with respect thereto. Such concepts may have applicability throughout the entire specification.

The previous description of the disclosed implementations is provided to enable any person skilled in the art to make or use the present invention. Various modifications to these implementations will be readily apparent to those skilled in the art, and the generic principles defined herein may be applied to other implementations without departing from the spirit or scope of the invention. Thus, the present invention is not intended to be limited to the implementations shown herein but is to be accorded the widest scope consistent with the principles and novel features disclosed herein.

What is claimed is:

1. A method for repairing tissue, the method comprising:
advancing a snare loop from a distal end of a body portion of a snare device such that a piercing element on a distal end of the snare loop pierces a tissue, wherein the snare loop comprises a monofilament;
advancing a fixation material through the snare loop; and
after advancing the fixation material through the snare loop, retracting the snare loop at least partially into the body portion of the snare device to capture the fixation material.

2. The method of claim 1, further comprising advancing the body portion of the snare device into a tunnel in a bone.

3. The method of claim 2, further comprising, after advancing the body portion of the snare device into the tunnel in the bone and retracting the snare loop at least partially into the body portion of the snare device to capture the fixation material, retracting the snare device to pull the fixation material through a tunnel in the bone.

4. The method of claim 1, wherein the piercing element comprises a plurality of ends of the monofilament twisted together.

5. The method of claim 1, wherein exposing the snare loop comprises extending the snare loop 8-10 cm beyond a distal end of the body portion of the snare device.

6. The method of claim 1, wherein retracting the snare loop to capture the fixation material comprises pulling the fixation material into the body portion of the snare device.

7. The method of claim 1, wherein the piercing element comprises a plurality of monofilaments, wherein a distal tip of each monofilament is welded together.

8. The method of claim 1, further comprising extending the snare loop through a tissue repair guide, the tissue repair guide configured to guide the snare loop.

9. The method of claim 1, wherein advancing the snare loop from the distal end of the body portion comprises moving a sliding actuator on the body portion.

10. The method of claim 1, wherein advancing the snare loop from the distal end of the body portion comprises moving a movable portion of the body portion with respect to a main portion of the body portion.

11. A method for repairing tissue, the method comprising:
advancing a snare loop from a distal end of a body portion of a snare device such that a piercing element on a distal end of the snare loop pierces a tissue;
advancing a suture through the snare loop; and
after advancing the suture through the snare loop, retracting the snare loop at least partially into the body portion of the snare device to capture the suture.

12. The method of claim 11, further comprising advancing the body portion of the snare device into a tunnel in a bone.

13. The method of claim 12, further comprising, after advancing the body portion of the snare device into the tunnel in the bone and retracting the snare loop at least partially into the body portion of the snare device to capture the suture, retracting the snare device to pull the suture through a tunnel in the bone.

14. The method of claim 11, wherein the piercing element comprises a plurality of monofilaments twisted together.

15. The method of claim 11, wherein exposing the snare loop comprises extending the snare loop 8-10 cm beyond a distal end of the body portion of the snare device.

16. The method of any claim 11, wherein retracting the snare loop to capture the suture comprises pulling the suture into the body portion of the snare device.

17. The method of claim 11, wherein the piercing element comprises a plurality of monofilaments, wherein a distal tip of each monofilament is welded together.

18. The method of claim 11, further comprising extending the snare loop through a tissue repair guide, the tissue repair guide configured to guide the snare loop.

19. The method of claim 11, wherein advancing the snare loop from the distal end of the body portion comprises moving a sliding actuator on the body portion.

20. The method of claim 11, wherein advancing the snare loop from the distal end of the body portion comprises moving a movable portion of the body portion with respect to a main portion of the body portion.
